(19) United States
(12) Patent Application Publication
(10) Pub. No.: US 2011/0218642 A1
(43) Pub. Date: Sep. 8, 2011

(54) MANIPULATION SYSTEM FOR SELECTION

(75) Inventor: Karl-Heinz Widmer, Merzhausen (DE)

(73) Assignee: IMPLANTECH MEDIZINTECHNIK GES.M.B.H., Maria Enzersdorf (AT)

(21) Appl. No.: 13/125,300

(22) PCT Filed: Oct. 23, 2009
(86) PCT No.: PCT/EP2009/063977

§ 371 Date: May 25, 2011

(30) Foreign Application Priority Data
Oct. 23, 2008 (AT) A 1663/2008

(51) Int. Cl. A61F 2/32

(52) U.S. Cl. 623/22.42

(57) ABSTRACT

The present invention relates to a navigation device (4) for visually selecting and positioning components of a multi-part joint endoprosthesis (1) for ball joints of a human or animal body, in particular a hip joint endoprosthesis, with a shaft (2) that can be inserted into a marrow cavity of a bone, an exchangeable connection neck (3) and a joint head located at the free end of the connection neck (3), forming a ball joint with a joint socket (5) that can be connected to a bone, wherein there is provided at least one navigation ball (4) with an outer diameter corresponding to the joint socket (5) and with a recess (8) for attachment at the free end of the connection neck (3) as well as with at least one partially peripheral marking (6) provided on its surface, as well as two or more connection necks (3) of different geometries, wherein each connection neck (3) may be connected with the shaft (2) at different positions and at the same or at different CCD angles and at the same or different antetorsion angles in regard to the shaft, respectively.

The invention relates to a manipulation system for visually selecting and positioning components of a multi-part joint endoprosthesis for ball joints of a human or animal body, in particular a hip joint endoprosthesis, with a shaft that can be inserted into a marrow cavity of a bone, an exchangeable connection neck and a joint head located at the free end of the connection neck, forming a ball joint with a joint socket that can be connected to a bone.

Endoprostheses for shoulder and hip joints have been successfully used in a variety of forms and embodiments as useful auxiliary elements in order to replace a damaged joint, either by an accident or due to degradation, such as arthrosis. In this case, there is usually inserted a fixation or shaft component into the marrow cavity of the appropriately prepared bone, continuing into another joint component, which then interacts with another prosthesis component inserted into the other bone of the joint to be provided with a prosthesis. Such an endoprosthesis for a hip joint is, for example, known from the WO 95/13757.

From DE 44 07 227 A1 there is further known a joint prosthesis, providing a controlled angular orientation of the neck section in regard to the shaft by means of a module-like configuration of the connection neck between the femoral stem and the femoral head.

A prosthesis with a similar configuration is also known from the WO 00/64384. This describes an endoprosthesis in the form of an implant, the ends of which are conically formed, which is inserted into the bone, with the implant consisting of two components. The one component is formed as a multi-spline profile with meandering or undulating elevations, allowing thus for an interlocking fixation in the insertion opening of complementary form, located in the femoral stem. The other component consists of a conical neck section with a smooth surface, at the free end of which there may be attached a hip head. The axis of this neck section is positioned in a defined angle to the axis of the end section, fixed in an interlocking wax in the femoral stem. By twisting the end section in the femoral stem in accordance with the elevations as well as by replacement of neck sections with various lengths, the prosthesis may be gradually adapted to the anatomical structures.

Furthermore, there is known from the DE 92 16 094 U1 a femoral component of a hip joint endoprosthesis, wherein the longitudinal axes of the two end sections with conical form are offset in parallel. In order to fix the position of the prosthesis, there are provided, accommodated in the femoral stem, attachment means like screws, or this end section is to be fixed in an interlocking way in the cavity of the femoral stem due to its special geometrical form. The free other end of the prosthesis projects into the corresponding conical cavity of the joint head.

The aim of reconstructing a ball joint, in particular a hip joint, must be to intra-operatively reconstitute the original range of motion as far as possible. Only upon achieving this aim, the danger of impingement (FAI, Femoroacetabular Impingement with hip joints; an osseous impingement between the joint-near part of the femoral bone and the joint socket) or dislocation will be eliminated. For a successful reconstruction of the ball joint, the individual adjustment of the CCD angle (abbreviation for caput-collum-diaphyseal angle; describes the angle between the femoral neck and the stem of the femoral bone) is of steadily growing importance.

Prior art prostheses have the disadvantage that they do not assist the surgeon in determining the optimal insertion position according to the individual anatomical structures of the patient.

From the EP 0 363 019 A2 there is known a test joint head with partly peripheral markings, which may be inserted into an artificial joint socket. The approximately semi-circular test joint head is at one side flattened out, projecting beyond the artificial joint socket upon insertion, in order to provide for easier handling.

This test joint head is disadvantageous as it cannot be connected with the neighbouring and subsequent, respectively, components of the joint endoprosthesis. Hence, it is not possible to determine whether a joint head, inserted instead of the test joint head later on, may be connected with a subsequent intermediate and connection section, respectively, in the optimal insertion position for each.

In the WO 2003/057087 A2 there is shown an auxiliary means for implanting a hip joint endoprosthesis, comprising a manipulation socket, a manipulation joint head as well as a device for maintaining the oriented position of the manipulation socket. By means of this device, it is then possible to orient appropriately a bone cutter and a drive-in instrument for positioning the hip socket.

This auxiliary means is an aligning jig for determining the oriented position of the manipulation socket in regard to its angle towards the acetabulum. This embodiment is insofar disadvantageous as, during surgery, there has to be screwed a guiding rod, onto which there is shifted an aligning jig, into the pelvic bone of the patient. The guiding rod will be surgically removed from the pelvic bone upon insertion of the joint endoprosthesis. Another disadvantage is that there is provided only a predetermined, not exchangeable prosthesis neck for attachment to the joint head. Here, an individual connection of the prosthesis neck with the joint stem with individually different antetorsion angles is not possible.

By means of computer assisted implantation, which require, however, rather complex apparatuses, it has recently been possible to perform the entire measurement and positioning procedures of all prosthesis components and, in this way, obtain an improved result in view of the range of motion. There have been used robots as navigation devices. [Widmer et. al.: “The impact of the CCD-angle on range of motion and cup positioning in total hip arthroplasty”. Clinical Biomechanics 20, (2005), p. 723-728]; [Haaker et. al.: “Comparison of Conventional Versus Computer-Navigated Acetabular Component Insertion”. J. Arthroplasty 22 (2), (2007), p. 151-159]

It is, however, disadvantageous that not all hospitals are equipped with such computer controlled robots for the measurement of body geometry and for computer assisted implantation, respectively, and that such procedures of measurement and operation are rather complex in terms of equipment as well time-consuming. It is further disadvantageous that the medical staff has to be given special trainings in order to operate these computer assisted chirurgical procedures.

Thus, it is the aim of the present invention to provide a simple visual aid for determining the optimal three-dimensional position of components of a multi-part joint endoprosthesis. The optimal positioning is typically specific for every single prosthesis. In this way, it should be made possible to obtain the best reconstruction possible of the damaged ball joint for the patient, even without rather complex apparatuses.

Another aim of the present invention is to prevent, as far as possible, by means of a visual evaluation and adjustment of the optimal position of the prosthesis, potential post-operative complications upon total replacement of a ball joint. In this way, subsequent costs due to further treatment, on the one side, should be minimized, and, on the other side, there will be made an essential contribution to the well-being of the patient.

This aim is solved according to the invention by making use of a manipulation system for visually selecting and positioning components of a multi-part joint endoprosthesis for ball joints of a human or animal body, in particular a hip joint endoprosthesis, with a shaft that can be inserted into a marrow cavity of a bone, an exchangeable connection neck and a joint head located at the free end of the connection neck, forming a ball joint with a joint socket that can be connected to a bone, characterized in that there is provided at least one navigation ball with an outer diameter corresponding to the joint socket and with a recess for attachment at the free end of the connection neck as well as with at least one partially peripheral marking provided on its surface, and that there are provided two or more connection necks of different geometries, wherein each connection neck may be connected with the shaft at different positions and at the same or at different CCD angles and at the same or different antetorsion angles in regard to the shaft, respectively.

The marking at the navigation ball thus also indicates by means of which connection neck there may be obtained the best reconstruction of the damaged joint possible.

In a preferred embodiment the model-like connection necks are formed in different geometries from a metallic material, as it is also used for the production of prostheses. The connection between the model-like connection necks and the shaft is designed so that it may be disconnected easily and if possible manually and so that a simple exchange and test of several connection necks is possible during the surgical procedure.

In another embodiment for solving the aim according to the invention, the manipulation system is characterized in that there are provided at least two navigation balls with different diameters, correlating with joint sockets of different geometries.

Advantageously, this manipulation system will provide the surgeon with numerous variants for visually selecting the optimal insertion position and insertion length of the connection neck as well as of the navigation ball for adjustment to the individual geometry of the joint to be replaced.

By means of the marking on the navigation ball, there is determined, during insertion, the actual state of the relative position of shaft and joint socket. This enables a visual assessment of inclination and anteversion of the socket as well as of the CCD angle and the antetorsion angle of the shaft.

In a manipulation system according to the invention, there are usefully provided at least two navigation balls with different diameters, correlating with joint sockets of different geometries.

Advantageously, the marking provided on the surface of the navigation ball in a manipulation system is situated in a marking plane, wherein this marking plane forms an angle of 50° to 130° with the axis of the connection neck.

The precise angle and the exact orientation, respectively, of the marking on the navigation ball is specific for every single prosthesis system, and this is evaluated and determined, respectively, for every single combination of connection neck and joint head of the prosthesis.

Such a marking is especially usefully arranged on the navigation ball in the inserted position in parallel to the edge of the joint socket, and it is preferably situated in the same plane as the socket.

In a variant of the invention, there are provided in a manipulation system markings on the navigation ball in two or more marking planes, which are in parallel to each other.

For an optimal relative position of the components of the joint endoprosthesis, the markings of the navigation ball may be arranged, in the inserted position, in parallel to the edge of the joint socket, and a marking, preferably a central marking, may for example be situated in the same plane as the socket. The markings in two or more marking planes may also be separated from each other by different colours or by recesses.

In a preferred embodiment of the invention, the markings in a manipulation system are formed on the surface of the navigation ball as grooves, in particular as notches, or as lines.

In another advantageous embodiment, there may also be provided several parallel markings on the navigation ball, with the markings on the surface of the navigation ball being preferably designed as follows:
as notch-like or engraved grooves,
as lines, in dotted, broken or continuous form,
as peripheral, bank-like surfaces that are separated by different colours,
arranged in one or more marking planes in parallel to each other.

Within the scope of the invention, however, it is also possible to use a manipulation system, in which the markings on the surface of the navigation ball are designed as peripheral colour bands separated by different colours.

In another development of the inventive manipulation system the markings on the surface of the navigation ball are designed as ball parts, in particular semi-spherical parts, of different colours, with the ball part of the navigation ball located outside of the joint socket, in the inserted position, in particular a half of the ball, being designed in another colour than the ball part situated, in the inserted position, within the joint socket. The marking is represented by the colours indicated alongside the periphery of the navigation ball.

Usefully in a manipulation system according to the invention, the markings on the surface of the navigation ball are designed as at least one recess, with the ball part of the navigation ball situated, in the inserted position, outside of the joint socket, in particular a half of the ball, having a smaller radius than the ball part situated, in the inserted position, within the joint socket, hence forming a recess.

The navigation ball in a manipulation system advantageously has a recess for attachment at the free end of a model-like connection neck, wherein by means of the appropriate form and shape of this recess, preferably by provision of tongue and groove, the rotational position of the navigation ball is fixed relative to the connection neck.

Preferably, the recess for attachment at the free end of the connection neck is adapted so that the navigation ball may be attached onto the connection neck easily and, if possible, manually and that it may also be disconnected therefrom. In a particular embodiment such a navigation ball is made from a plastic material, which is usually also used for the production of prostheses and has also been tested for its compatibility with the human organism.

Furthermore, the invention provides an instruction manual for advantageously selecting and positioning a multi-part joint endoprosthesis for ball joints. This instruction manual describes the optimal course of events for the optimal positioning of the components of the joint endoprosthesis in the reconstruction of the damaged ball joint of a human or animal body.

In this way, there are first established the connections of the multi-part joint endoprosthesis with the neighbouring bones in a way so that a shaft is inserted into the marrow cavity in the one bone—in the case of a hip joint endoprosthesis into the femoral bone—in an interlocking way. Instead of the joint socket, there is inserted and attached, respectively, an artificial joint socket at the respective bone—in the case of a hip joint endprosthesis at the hip bone.

Instead of the damaged neck including joint head of the femoral bone, there is now selected an artificial, model-like connection neck from the manipulation system as a surrogate for the later to be inserted and final connection neck, and this surrogate is connected with the already implanted shaft.

Subsequently, there is inserted a navigation ball, the diameter of which has been selected in advance as suitable for the geometry of the inserted joint socket, into the recess provided therefore at the model-like connection neck and introduced into the joint socket. This navigation ball also serves as a kind of surrogate or sample for the joint head, by which it will later on be replaced. On the basis of the marking on the navigation ball, there may then be determined visually the insertion position of the connection neck in regard to the position of the shaft and in regard to the position of the joint socket, respectively.

Upon disconnecting the attachment between shaft and connection neck, the model-like connection neck may be re-inserted into the shaft in an altered position, or the model-like connection neck is removed and replaced by another model-like connection neck of different geometry, also provided in a manipulation system. In this way, it is possible to vary the length of the connection neck as well as the angular position in regard to the shaft. Hence, the free end of the connection neck projecting into the navigation ball may be navigated in its spatial position.

This procedure of selection is performed until the marking on the navigation ball, in a preferred embodiment until one of several, and preferably a central one of the markings arranged in parallel on the navigation ball, rests with the edge of the joint socket in a planar arrangement. If this position is obtained, the surgeon will recognize that the positioning of the connection neck and the model-like navigation ball in regard to shaft and joint socket of the ball joint has been realized in an optimal way and that the adaptation of the prosthesis to the individual anatomical conditions is as optimal as possible.

Finally, the model-like connection neck, which is replaceably attached in the shaft, is replaced by the final connection neck with same geometry. In a preferred embodiment, the final connection neck is fabricated in a more resilient material of higher quality than the model-like connection necks available in the manipulation system. The final connection neck is then sufficiently stably connected with the shaft.

The navigation ball, which has also served as a surrogate during the surgical procedure, is then replaced by a final joint head with same geometry, and the joint head is then attached at the free end of the connection neck instead of the navigation ball. Also the final joint head is preferably made from another material of higher quality than the navigation ball. Inbetween the final connection neck and the joint head there is provided, by driving in the joint head, a sufficiently stable connection.

Connection neck and joint head remain as essential components of the reconstructed joint endoprosthesis in the body in the inserted and beforehand optimized position. The surgical procedure thus may be continued and terminated.

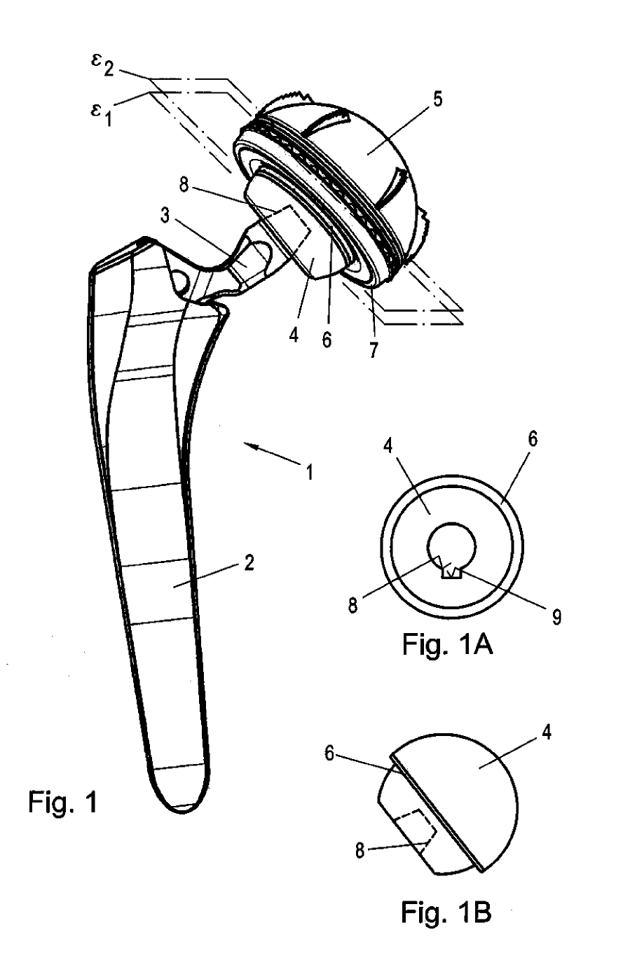

An exemplary embodiment of a manipulation system designed according to the invention for the optimal positioning of a multi-part endoprosthesis is illustrated in the FIGS. 1 to 4 and further explained in the following description, wherein the FIGS. 1 to 4 each show a schematical perspective thereof. FIG. 1A shows in detail a schematic view of a navigation ball from the side of the neck. FIG. 1B shows in a schematic side view a navigation ball with a particular embodiment of the marking in the form of a recess.

FIG. 1 shows in a schematic perspective depiction an exemplary embodiment of a joint prosthesis 1 already implanted in a human body, wherein, however, the human body parts such as e.g., bones are not depicted, which is in the exemplary embodiment formed as a hip joint endoprosthesis 1 for the provision of a prosthesis in the human hip joint. The joint endoprosthesis 1 is formed as a multi-part element and contains a shaft 2 with a model-like connection neck 3, which may be attached therein in different positions and which may be inserted at one end. The connection neck 3 projects with its other preferably conically formed end into the corresponding provided recess 8 of the inventive navigation ball 4, which is also model-like inserted during the surgical procedure and is later on replaced by and exchanged for another, not depicted, ball joint head with the same geometry in the optimal position of the joint. The navigation ball 4 projects into a joint socket 5 and therein ideally meets the joint socket in the centre. As depicted in FIG. 1, there are provided several parallel markings 6 on the navigation ball 4. The two marking planes ε₁, ε₂ that may be seen in FIG. 1 are arranged in parallel to the edge 7 of the joint socket 5, with the central one of the preferably three marking planes ε₁, ε₂, ε₃ being situated in the plane of the edge 7 of the joint socket 5. In use of such a prosthesis-specific navigation head, the expert will recognize in view of a neutrally adjusted joint that the CCD angle and the total antetorsion angle are precise and that thus the joint endoprosthesis is positioned in an ideal way.

In FIG. 1A there is displayed in a detail view from the neck side a navigation ball 4. There may be seen the peripheral markings 6 as well as the recess 8, corresponding with the connection neck 3 not depicted in FIG. 1A. The recess 8 herein is configured in a tongue-groove form 9, by means of which the rotational position of the navigation ball 4 is fixed in regard to the connection neck 3.

FIG. 1B illustrates a navigation ball 4 in another detailed view from the side. In this special case, the marking 6 is formed as a recess between the ball part of larger radius, which is, in the inserted position, situated within the joint socket, and the ball part of smaller radius, which is, in the inserted position, situated outside of the joint socket. In FIG. 1B the recess 8 for the accommodation of the connection neck 3, herein not displayed, is shown in a broken line.

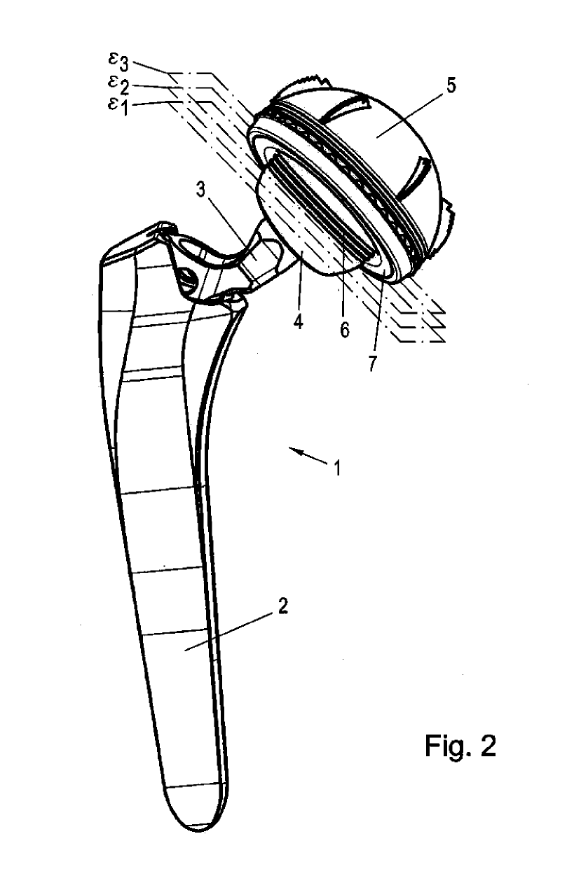

In FIG. 2 the central of several markings 6 is located on the navigation ball 4 in the middle of the surface of the navigation ball 4 that is facing the observer, in a great distance from the edge 7 of the joint socket 5, the planes of the markings ε₁, ε₂, ε₃ are inclined to the front towards the viewer, and they are not arranged in parallel but rather at an acute angle to the plane of the edge 7 of the joint socket 5. Here, those skilled in the art will realize that the insertion position of the connection neck 3 and hence the antetorsion angle have to be adapted accordingly, or that there has to be chosen another model-like connection neck 3 from the manipulation system, in order to obtain the ideal condition, as depicted in FIG. 1. The CCD angle in FIG. 2 is adjusted to be more or less parallel and satisfactory.

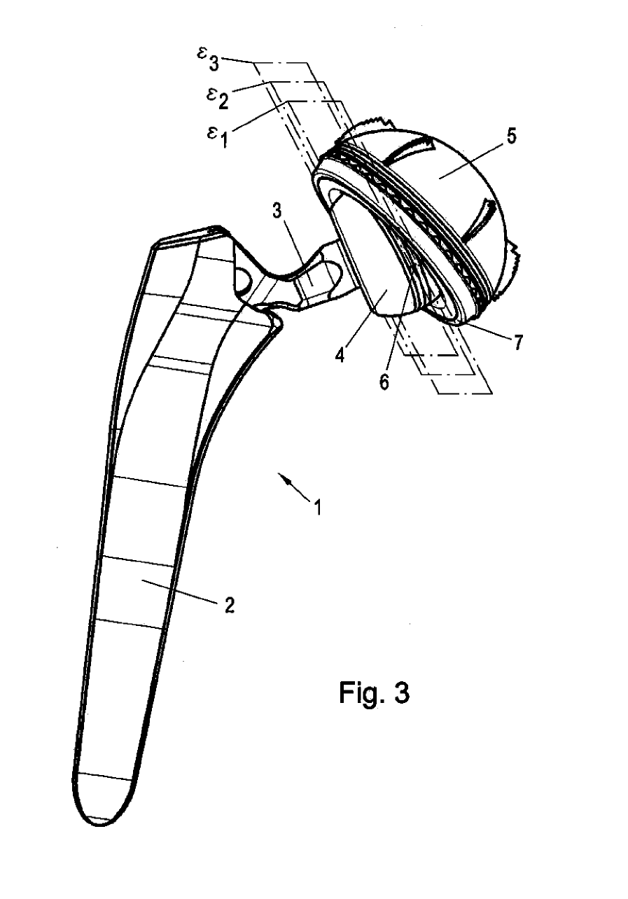

In FIG. 3, the central marking 6 on the navigation ball 4 in the middle of the surface of the navigation ball 4 facing the viewer is covered by the edge 7 of the joint socket 5, and in this way the expert will recognize that the correct antetorsion angle has been obtained. The marking planes ε₁, ε₂, ε₃ of the markings 6 on the navigation ball 4, however, are not in parallel to the plane of the edge 7 of the joint socket 5. For this reason, there has to be chosen another model-like connection neck 3 with modified CCD angle, in order to obtain the ideal condition, as depicted in FIG. 1.

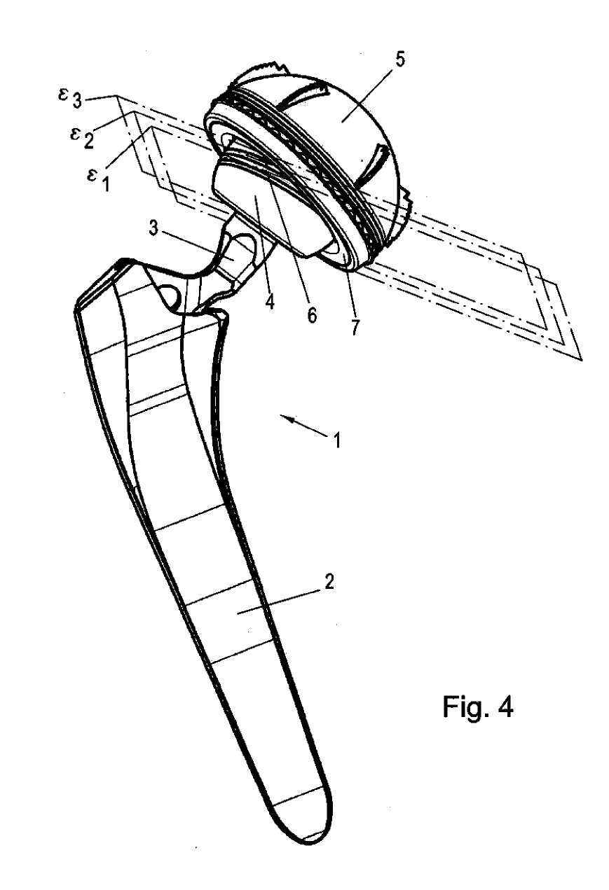

In FIG. 4 the central marking 6 on the navigation ball 4 again is covered in the middle of the surface of the navigation ball 4 facing the viewer by the edge 7 of the joint socket 5. As in FIG. 3, also in FIG. 4 the selected antetorsion angle is correct. The marking planes ε₁, ε₂, ε₃ in FIG. 4 are not in parallel to the plane of the edge 7 of the joint socket 5. Hence, there also has to be chosen another model-like connection neck 3 with modified CCD angle in order to obtain the ideal insertion position according to FIG. 1.

1. A manipulation system for visually selecting and positioning components of a multi-part joint endoprosthesis for ball joints of a human or animal body, in particular a hip joint endoprosthesis, with a shaft (2) that can be inserted into a marrow cavity of a bone, an exchangeable connection neck (3) and a joint head located at the free end of the connection neck (3), forming a ball joint with a joint socket (5) that can be connected to a bone,
characterized in that
there is provided at least one navigation ball (4) with an outer diameter corresponding to the joint socket (5) and with a recess (8) for attachment at the free end of the connection neck (3) as well as with at least one partially peripheral marking (6) provided on its surface, as well as two or more connection necks (3) of different geometries, wherein each connection neck (3) may be connected with the shaft (2) at different positions and at the same or at different CCD angles and at the same or different antetorsion angles in regard to the shaft, respectively.

2. A manipulation system according to claim 1, characterized in that there are provided two or several model-like connection necks (3) of different geometries.

3. A manipulation system according to claim 1 or 2, characterized in that there are provided at least two navigation balls (4) with different diameters, correlating with the joint sockets (5) of different geometries.

4. A manipulation system according to any of the claims 1 to 3, characterized in that the marking (6) provided at the surface of the navigation ball (4) is located in a marking plane (ε₁), with this marking plane (ε₁) forming an angle of between 50° to 130° with the axis of the connection neck (3).

5. A manipulation system according to any of the claims 1 to 4, characterized in that the marking (6) on the navigation ball (4) is arranged, in the inserted position, in parallel to the edge (7) of the joint socket (5) and that it is preferably situated in the same plane therewith.

6. A manipulation system according to claim 5, characterized in that there are provided markings (6) on the navigation ball (4) in two or several marking planes (εᵢ=1 to n) in parallel to each other.

7. A manipulation system according to any of the claims 1 to 6, characterized in that the markings (6) on the surface of the navigation ball (4) are formed as grooves, in particular as notches, or as lines.

8. A manipulation system according to any of the claims 1 to 7, characterized in that the markings (6) on the surface of the navigation ball (4) are formed as peripheral colour bands separated by colour.

9. A manipulation system according to any of the claims 1 to 7, characterized in that the markings (6) on the surface of the navigation ball (4) are designed as ball parts of different colour, in particular as semi-spherical parts, with the ball part of the navigation ball (4), in particular one half of the ball, situated, in the inserted position, outside of the joint socket (5), configured in another colour than the ball part situated, in the inserted position, within the joint socket (5).

10. A manipulation system according to any of the claims 1 to 9, characterized in that the markings (6) on the surface of the navigation ball (4) are provided at least as one recess, with the ball part of the navigation ball (4), in particular one half of the ball, situated, in the inserted position, outside of the joint socket (5), having a smaller radius than the ball part situated, in the inserted position, inside the joint socket (5), in this way forming a recess.

11. A manipulation system according to any of the claims 1 to 10, characterized in that the navigation ball (4) has a recess (8) for attachment at the free end of a model-like connection neck (3), wherein by means of the appropriate form and shape of this recess (8), preferably by provision of tongue and groove, the rotational position of the navigation ball is fixed relative to the connection neck (3).
